SEQUENCE LISTING (6 sequences)

SEQ ID NO: 1 - protein, 27 aa, artificial
  Fragment of human protein
CQITWFKNNHKIQQEPGIILGPGSSTD

SEQ ID NO: 2 - protein, 28 aa, artificial
  Fragment of human protein
HKIILGPGSSTLFIERVTEEDEGVYHCK

SEQ ID NO: 3 - protein, 29 aa, artificial
  Fragment of human protein
KMEKRLHAVPAANTVEFVCKVYSDAQPHI

SEQ ID NO: 4 - protein, 28 aa, artificial
  Fragment of human protein
DRIYISANRQLCYHHSLNWTKVLRGPTD

SEQ ID NO: 5 - protein, 30 aa, artificial
  Fragment of human protein
WLSAWTNDAMADSRQNNTSLRLGVYAALCH

SEQ ID NO: 6 - protein, 27 aa, artificial
  Fragment of human protein
GLSLDASMHSQLRILDSKFRRTRPLEC

(19) United States
(12) Patent Application Publication
(10) Pub. No.: US 2019/0204326 A1
(43) Pub. Date: Jul. 4, 2019

(54) ANTIGEN BIOMARKERS

(71) Applicant: The University of the Highlands and Islands, Inverness (GB)

(72) Inventor: Jun Wei, Inverness (GB)

(21) Appl. No.: 16/330,275

(22) PCT Filed: Sep. 27, 2017
(86) PCT No.: PCT/GB2017/052879

§ 371 Date: Mar. 4, 2019

(30) Foreign Application Priority Data
Sep. 27, 2016 (GB) 1616357.8
Apr. 13, 2017 (GB) 1705988.2

(51) Int. Cl. G01N 33/574; A61P 35/00; C07K 16/06; C07K 16/28; C07K 16/32

(52) U.S. Cl. CPC G01N 33/57484; A61P 35/00; C07K 16/32; C07K 16/2863; C07K 16/065

(57) ABSTRACT

Peptide antigens are provided that may be used to screen blood plasma for antibodies that be used as anticancer therapeutics. Kits comprising the peptide antigens, and methods of using the same are also provided.

### FIELD OF THE INVENTION

The invention relates to antigen biomarkers and their use in the screening of antibodies for therapeutic activity, specifically screening of antibodies for anticancer therapeutic activity.

### BACKGROUND OF THE INVENTION

Cancer is a prominent disease that is having an increasing impact on the human population. Based on the latest global cancer statistics, 14.1 million new cases and 8.2 million deaths occurred in 2012 worldwide (Torre et al., CA Cancer J Clin 65: 87-108, 2015). The occurrence of cancer is increasing due to the growth and aging of the population.

Whilst survival rates are increasing for at least some types of cancers, these improvements are at least due in part to the increase in the number of cases that are being diagnosed early where the cancer has not grown or spread to a dangerous degree, and can therefore be more successfully treated by medication and/or surgery.

Many of the available treatments for cancer are cytotoxic treatments wherein the clinician attempts to target and kill the cancer cells within the patient with medication or radiation, for example. However, such cytotoxic agents are often cytotoxic to the native healthy cells as well to the cancer cells, and can result in significant side effects for the patient. In addition, the maximum dosage of the cytotoxic agent that can be given to a patient is often lower than may be optimum for killing the target cancer cells, as the effect of higher doses of the cytotoxic agent would have too high a toll on the healthy cells of the patient to be acceptable.

Tumour cells have the capability to produce some growth factors such as vascular endothelial growth factors (VEGFs) and epidermal growth factor receptors (EGFRs), which can bind to their corresponding receptors on the surfaces of cells, resulting in a variety of biological effects and thereby promoting tumour progression (Scartozzi M, et al: PLOS One 7: e38192, 2012). It has also been shown that Hsp90, EGFR, VEGF and Protein Kinase B (AKT) are known to play a role in radiation resistance (Sheridan MT, et al.: Radiat Oncol Investig 5: 180-186, 1997; Tanno S, et al.: Cancer Res 64: 3486-3490, 2004.), and radiation exposure may result in activation of EGFR, which can in turn activate the phosphatidylinositol-4,5-bisphosphate 3-kinase(P13K)/AKT and signal transducer and activator of transcription 3 (STAT3) pathways, and upregulate VEGF production (Bowers G, et al.: Oncogene 20: 1388-1397, 2001.).

Natural antibodies have been known for nearly half a century. Despite knowledge about the role of the polyreactive natural IgM in pathogen elimination, natural antibodies also play a role in the maintenance of homeostasis, inflammatory diseases, autoimmunity and anti-tumor cytotoxicity (Boehm et al, Gerontology 56:303-309, 2010; Schwartz-Albiez et al, Autoimmun Rev 7: 491-495, 2008). IgG antibody in the circulation has been thought to be a serological hallmark of autoimmune diseases, but an increasing number of studies have also revealed a link between autoantibodies and many non-autoimmune conditions such as cancer and neurological disease (Eric et al, PLOS ONE 8: e60726, 2013).

Compositions comprising gamma-globulins derived from human antibodies have been used for the treatment of a number of diseases. However, there remains a need for the effective identification of human sources of therapeutically effective antibodies that may be processed into gamma-globulin treatments for cancer.

Therefore, it is an object of the present invention to provide methods and targets for the identification of useful sources of antibodies that may be therapeutically effective against cancer, and compositions comprising said antibodies.

### SUMMARY OF THE INVENTION

According to a first aspect of the invention there is provided a method of determining whether a biological sample comprises target antibodies that have anticancer activity, the method comprising the steps:
i) providing a biological sample;
ii) providing at least one peptide antigen according to any one of SEQ ID NO:1-SEQ ID NO:6 or functional variants thereof;
iii) contacting the biological sample to the at least one peptide antigens; and
iv) determining the concentration of target antibodies that were present in the biological sample that bind specifically to the peptide antigens; and
v) comparing the determined concentration of target antibodies present in the biological sample to a reference concentration,
   wherein a significant increase in the concentration of target antibodies in the biological sample compared to the reference concentration is indicative that the biological sample comprises a significant concentration of the one or more target antibodies that have anticancer activity.

TABLE 1
Peptide antigens according to the invention

| SEQ | | Target |
|---|---|---|
| ID NO: | Peptide Sequence | Protein |
| 1 | CQITWFKNNHKIQQEPGIILGPGSSTD | VEGFR1a |
| 2 | HKIILGPGSSTLFIERVTEEDEGVYHCK | VEGFR1b |
| 3 | KMEKRLHAVPAANTVEFVCKVYSDAQPHI | FGFR2 |
| 4 | DRIYISANRQLCYHHSLNVVTKVLRGPTD | ERBB3 |
| 5 | WLSAWTNDAMADSRQNNTSLRLGVYAALCH | ABCC3 |
| 6 | GLSLDASMHSQLRILDSKFRRTRPLEC | ABCC5 |

The or each peptide antigen or functional variants thereof may correspond to a peptide sequence of a protein that is the target of a target antibody. Accordingly, the protein may be a target protein. The peptide sequence may not directly correspond to a sequence of the target protein, but may correspond to or mimic the arrangement of peptides of the folded target protein that form the binding site of the target antibody.

The or each peptide antigen or functional variants thereof may be adapted to selectively bind to an antibody that binds to a cell membrane protein. Accordingly, the cell membrane protein may be the target protein. The peptide antigen or functional variants thereof may be derived from a cell membrane protein that is highly expressed in common cancer cells. The peptide antigen or functional variants thereof may be adapted to selectively bind to an antibody that binds to a cell membrane protein that is highly expressed in common cancer cells. For example, the peptide antigen or functional variants thereof may be adapted to selectively bind to an antibody that binds to a cell membrane protein that is highly expressed in liver cancer cells, lung cancer cells, stomach cancer cells, pancreatic cancer cells, or esophageal cancer cells. The cell membrane protein may be highly expressed in two or more types of cancer cells. The cell membrane protein may be highly expressed in the majority of types of cancer cells.

For example, Table 2 shows examples of cancer tissues that highly express the target proteins of the peptide antigens on their surface.

TABLE 2
Cancer cells that highly express the target proteins listed in Table 1

| Target Protein | Cancer tissue | Comments |
|---|---|---|
| VEGFR1a | Brain, kidney, liver, | More than one isoform |
| | leukaemia & lymphoma | |
| VEGFR1b | Brain, kidney, liver, | More than one isoform |
| | leukaemia & lymphoma | |
| FGFR2 | Kidney, liver, sarcoma & colon | More than one isoform |
| ERBB3 | Bladder, breast ovarian, | More than one isoform |
| | colon & melanoma | |
| ABCC3 | Breast, esophagus, | |
| | head/neck, kidney, lung, | |
| | lymphoma & brain | |
| ABCC5 | Breast, colon, head/neck, | |
| | liver, lung, cervix & lymphoma | |

Typically, the target antibody does not bind to cell membrane proteins of healthy cells.

The peptide antigens according to any one of SEQ ID NO:1-SEQ ID NO:6 may be anchored to a substrate. The substrate may be suitable for use in an immunoassay. The substrate may be a planar substrate such as a glass or plastic slide or similar, and the peptide antigens may be bound to one or both planar surfaces of the planar substrate. The substrate may be a reaction vessel or a wall of a reaction vessel. The substrate may be a well. The substrate may be a well plate and the peptide antigens may be bound to the surface of one or more wells of the well plate. The substrate may be a particle. Accordingly the peptide antigens may be bound to the surface of the particle. The particle may be a bead or similar. The particle may be an aggregate, or a crystalline material.

In embodiments of the invention, the peptide antigens are preferably anchored to the surface of the substrate in such a way as to be available to specifically bind to the antibody. For example, the peptide antigens may be bound to the surface at the N- or C-terminus of the peptide antigen.

Alternatively, the peptide antigen may be bound to the surface via a linker. The linker may be a saturated or unsaturated hydrocarbon chain, an ether, a polymer, a polyethylglycol (PEG), a poly glycol, a poly ether or similar. The linker may be a peptide. In embodiments where the linker is a peptide, the peptide may be 1-10 amino acids in length, 1-20 amino acids in length, or 1-30 amino acids in length.

The linker may extend from the N-terminus of the peptide antigen. The linker may extend from the C-terminus of the peptide antigen.

The linker may comprise a binding group that allows the linker to bind the peptide antigen to the substrate. For example, in embodiments where the substrate is a silica substrate, the binding group may be a silane, siloxide, siloxane, or silanol, whereby the silicon-bearing group binds to the silica substrate.

The surface of the substrate that does not comprise peptide antigens may comprise a blocking agent to prevent or reduce non-specific binding of species that may be present in the biological sample to the substrate surface. For example, the surface may comprise a blocking protein that adsorbs to the substrate and blocks the substrate from other proteins that may be present in the biological sample. In another example, the surface may comprise a self-assembled monolayer (SAM). The SAM may comprise molecules having a binding head group and a tail group that inhibits or prevents non-specific binding. For example, in embodiments where the substrate is a glass substrate, the SAM may comprise an organo silane that binds to the silica of the substrate and the organic tail extends away from the surface.

The method may be an immunoassay. The immunoassay may use a label to detect target antibodies in a biological sample. Accordingly, the method may comprise the step of applying a label to target antibodies that have specifically bound to the peptide antigens. For example, the label may specifically bind to the target antibodies. The label may be any suitable detectable label known in the art. For example, the label may be an enzyme, a radioactive isotope, a DNA reporter, a fluorogenic reporter, or an electroluminescent tag.

The immunoassay may be a competitive assay system. The immunoassay may be a non-competitive assay system. For example, the immunoassay may use techniques such as immunocytochemistry, immunohistochemistry, radioimmunoassays, enzyme-linked immunosorbent assay (ELISA), “sandwich” immunoassays, precipitin reactions, gel diffusion precipitin reactions, immunodiffusion assays, agglutination assays, complement fixation assays, immunoradiometric assays, fluorescent immunoassays and the like.

In embodiments where a label is used to detect target antibodies, the label may be conjugated to a secondary antibody. The secondary antibody may be configured to bind to an epitope of target (“primary”) antibodies that have bound specifically to the peptide antigens. Accordingly, the method may comprise the step of contacting a labelled conjugate to target antibodies to thereby label the target antibodies. As a result, the label may allow the concentration of target antibodies to be determined from the concentration of label present.

The method may include the step of adding an antibody-enzyme conjugate that is configured to bind to any antibodies that have bound to the peptide antigen. In embodiments where the biological sample originates from a human subject, the method may include the step of adding an antihuman immunoglobulin (Ig). The antihuman Ig may be conjugated with an enzyme. The conjugate may be contacted with target antibodies that have bound to the peptide antigens. The method may further comprise the step of adding an enzyme substrate for the conjugated enzyme. Preferably, the action of the enzyme on the enzyme substrate induces a change in the enzyme substrate that is detectable. The enzyme may cleave the substrate to induce a detectable change in the substrate. The enzyme may oxidise the substrate to induce a detectable change in the substrate. For example, the conjugated enzyme may be a peroxidase and the enzyme substrate may produce a change in colour when the enzyme substrate is oxidised by the peroxidase. The peroxidase may be horse radish peroxidase (HRP). The substrate may be 3,3′,5,5′-tetramethylbenzidine (TMB). Other examples may be used and are well known to the person skilled in the art.

Accordingly, the method may be an enzyme-linked immunosorbent assay (ELISA).

In some embodiments a tertiary antibody may be added to bind to the secondary antibody. The tertiary antibody may comprise a moiety that interacts with the secondary antibody to thereby allow the secondary antibody, and thereby the target antibody, to be detected.

The method may include the step of washing the substrate after the application of a label to remove label that is not applied or bound to a target antibody.

The immunoassay may be a label free immunoassay. For example, the immunoassay may be a surface plasmon resonance assay where binding of the target antibody to the peptide antigen is directly detected.

The method may include the step of washing the biological sample from the peptide antigen after the step of contacting the biological sample to the peptide antigen. Accordingly, at least the majority of species that are not specifically bound to the peptide antigens may be removed.

Therefore, the method of the invention may determine whether a biological sample comprises a significant concentration of target antibodies that may potentially be used as anticancer therapeutics.

The biological sample may be a bodily fluid. Typically, the biological sample is a blood sample, such as a sample of whole blood or a sample of a blood fraction, such as blood plasma, or blood serum, for example. Alternatively, the biological sample may be lymphatic fluid, peritoneal fluid, cerebrospinal fluid or pleural fluid.

Typically, the biological sample is taken from a human subject prior to the method of the invention.

Biological samples identified using the method of the invention may be processed to purify, extract or amplify the target antibodies therein. For example, the biological sample may be processed to produce a gamma-globulin therapeutic.

The term “antibody” is used in the broadest sense and specifically covers monoclonal antibodies (including full length antibodies which have an immunoglobulin Fc region), antibody compositions with polyepitopic specificity, bispecific antibodies, diabodies, and single-chain molecules, as well as antibody fragments (e.g., Fab, F(ab′)₂, and Fv).

The basic 4-chain antibody unit is a heterotetrameric glycoprotein composed of two identical light (L) chains and two identical heavy (H) chains. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four CH domains for p and isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ϵ, γ and μ, respectively. The γ and μ classes are further divided into subclasses on the basis of relatively minor differences in the CH sequence and function, e.g., humans express the following subclasses: IgG1, IgG2, IgG3, IgG4, IgA1 and IgA2.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, the V regions consist of relatively invariant stretches called framework regions (FRs) of about 15-30 amino acid residues separated by shorter regions of extreme variability called “hyper-variable regions” or sometimes “complementarity determining regions” (CDRs) that are each approximately 9-12 amino acid residues in length. The variable domains of native heavy and light chains each comprise four FRs, largely adopting a p-sheet configuration, connected by three hyper-variable regions, which form loops connecting, and in some cases forming part of, the β-sheet structure. The hypervariable regions in each chain are held together in close proximity by the FRs and, with the hypervariable regions from the other chain, contribute to the formation of the antigen binding site of antibodies (see Kabat et al., Sequences of Proteins of Immunological Interest, 5th Ed. Public Health Service, National Institutes of Health, Bethesda, Md. (1991). The constant domains are not involved directly in binding an antibody to an antigen, but exhibit various effector functions, such as participation of the antibody dependent cellular cytotoxicity (ADCC).

“Functional variants” of a peptide such as the antigen used in the method of the invention, as used herein, includes a sequence resulting when a peptide is modified by, or at, one or more amino acids and retains the function of the specified peptide to the same degree, or to a sufficient degree for the method of the invention to be successfully carried out.

“Functional” as used herein refers to the ability of a peptide to bind to a target antibody with the same or similar affinity as the specific peptides listed above. Each peptide of SEQ ID NO:1 to SEQ ID NO:6 bind specifically to target antibodies. For example, the dissociation constant K_{D} of a peptide antigen to the target antibody may be less than 1×10⁻⁷, less than 1×10⁻⁸, less than 1×10⁻⁹ or less than 1×10⁻¹⁹. Accordingly, a functional variant of a given peptide antigen used in the method of the invention has an equilibrium dissociation constant that is at least 10% or 1% of the equilibrium constant of that peptide antigen. Methods that can be used to determine whether an antigen binds a specific antibody selectively are known in the art.

Some performance drop in a given property of variants may of course be tolerated, but the variants should retain suitable properties for the relevant application for which they are intended. Screening of variants of SEQ ID NO: 1 to SEQ ID NO: 6 can be used to identify whether they retain appropriate properties.

“Variants” of a peptide such as the peptide antigen of the invention, as used herein, includes a sequence resulting when a peptide is modified by, or at, one or more amino acids (for example 1, 2, 5 or even up to 10 amino acids if the substitutions are conservative substitutions as defined below).

The variant may have “conservative” substitutions, wherein a substituted amino acid has similar structural or chemical properties to the amino acid that replaces it, for example, replacement of leucine with isoleucine. A variant may have “non-conservative” changes, for example, replacement of a glycine with a tryptophan. Variants may also include sequences with amino acid deletions or insertions, or both. Guidance in determining which amino acid residues may be substituted, inserted, or deleted without abolishing the activity of the protein may be found using computer programs well known in the art.

In one example, one conservative substitution is included in the peptide, such as a conservative substitution in SEQ ID NO:1 to SEQ ID NO:6. In another example, 10 or fewer conservative substitutions are included in the peptide, such as five or fewer. A peptide of the invention may therefore include 1, 2, 3, 4, 5, 6, 7, 8, 9, 10 or more conservative substitutions. A peptide can be produced to contain one or more conservative substitutions by manipulating the nucleotide sequence that encodes that peptide using, for example, standard procedures such as site-directed mutagenesis or PCR. Alternatively, a peptide can be produced to contain one or more conservative substitutions by using peptide synthesis methods, for example, as known in the art.

Examples of amino acids which may be substituted for an original amino acid in a protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

In one embodiment, the substitutions are among Ala, Val, Leu and Ile; among Ser and Thr; among Asp and Glu; among Asn and Gln; among Lys and Arg; and/or among Phe and Tyr.

Further information about conservative substitutions can be found in, among other locations, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

A variant includes a “modified peptide” or “mutated peptide” which encompasses peptides having at least one substitution, insertion, and/or deletion of an amino acid. A modified or mutated peptide may have 1, 2, 3, 4, 5, 6, 7, 8, 9 or 10 or more amino acid modifications (selected from substitutions, insertions, deletions and combinations thereof).

Accordingly, fragments of the peptide antigens of any one of SEQ ID NOs: 1-6 that retain the function of selectively binding to the target antibody are included in the scope of the invention. For example, the peptide antigen fragment may comprise at least 10 amino acids of the peptide antigens of any one of SEQ ID NOs: 1-6. The peptide antigen fragment may comprise at least 15 amino acids of the peptide antigens of any one of SEQ ID NOs: 1-6. The peptide antigen fragment may comprise at least 20 amino acids of the peptide antigens of any one of SEQ ID NOs: 1-6.

By the term “significant increase” we refer to an increase in the concentration of the target antibody of at least 50%, at least 100%, at least 150% or at least 200% when compared to the average concentration of the same target antibody occurring in subjects that do not comprise a significant level or concentration of the or each target antibody. A significant level may be determined by a threshold value above which the concentration of the target antibody is determined to be above the noise level of the assay used and thereby be determined to be a positive result. The threshold value may be determined as a function of the standard deviation of the measured concentration of the or each target antibody in biological samples that do not have a significant concentration of the or each target antibody. The threshold value may be a concentration above which the or each target antibody may be extracted and/or purified.

The reference concentration of the target antibody may be the concentration of the target antibody in biological sample from a subject that does not express significant concentrations of the or each target antibody. The reference concentration of the target antibody may be a predetermined threshold value, i.e. a cut-off value. The cut-off value may be a percentile of determined concentrations from biological samples. For example, the cut-off value may be the 70^{th}, 80^{th}, 90^{th}, 95^{th}, 97.5^{th} percentile, above which a concentration is determined to be a significant concentration.

The invention extends in a second aspect to a peptide antigen corresponding to SEQ ID NOs:1 to 6 or functional variants thereof.

The peptide antigen may be used to detect antibodies of interest in a biological sample. The biological sample may be a bodily fluid. Typically, the biological sample is a blood sample, such as a sample of whole blood or a sample of a blood fraction, such as blood plasma, or blood serum, for example. Alternatively, the biological sample may be lymphatic fluid, peritoneal fluid, cerebrospinal fluid or pleural fluid.

The peptide antigen may be used in the method of the first aspect.

The peptide antigen may comprise a linker or spacer moiety that allows the peptide antigen to be anchored to a surface, whilst retaining the ability to bind to the target antibody.

The peptide antigen may be used to produce antibodies. The peptide antigen may be used to produce antibody fragments. The antibodies may be monoclonal antibodies. The antibodies produced may be used to treat cancer in patients. Typically, the antibodies are IgG antibodies. For example, the antibodies may be human IgG antibodies.

The peptide antigen may be used to produce antibody-like proteins. For example, the peptide antigen may be used to produce aptamers, or the like.

The antibodies or aptamers raised against the peptide antigens may have activity against abnormal cells such as cancer cells. For example, the antibodies or aptamers may bind to surface proteins in the cell membrane of an abnormal cell. Antibodies or aptamers bound to the cell membrane of abnormal cells may inhibit proliferation of the abnormal cells. Accordingly, the antibodies or aptamers may be used to inhibit proliferation of abnormal cells.

According to a third aspect of the invention there are provided antibodies or aptamers raised against the peptide antigens of SEQ ID NOs: 1-6 or functional variants thereof. The antibodies may be monoclonal antibodies. The antibodies produced may be used to treat cancer in patients. Typically, the antibodies are IgG antibodies. For example, the antibodies may be human IgG antibodies.

The antibodies may be whole antibodies. The antibodies may be fragments of antibodies. Typically, the antibodies comprise at least the specific variable domain fragment that binds to the specific peptide antigen of SEQ ID NOs: 1-6 or functional variants thereof.

Suitable methods of generating suitable antibodies or aptamers according to the present aspect and determining whether they specifically bind to the peptide antigens of the invention are well known in the art, and include methods such as using phage display methods such as those described in McCafferty, J.; Griffiths, A.; Winter, G.; Chiswell, D. (1990). “Phage antibodies: filamentous phage displaying antibody variable domains”. Nature. 348 (6301): 552-554.

The antibodies or aptamers may have activity against abnormal cells such as cancer cells. For example, the antibodies or aptamers may bind to surface proteins in the cell membrane of an abnormal cell. Antibodies or aptamers bound to the cell membrane of abnormal cells may inhibit proliferation of the abnormal cells. Accordingly, the antibodies or aptamers may be used to inhibit proliferation of abnormal cells.

According to a fourth aspect of the invention, there is provided a kit of parts comprising one or more peptide antigens according to SEQ ID NOs: 1-6 or functional variants thereof.

The kit of parts may comprise two or more antigens according to SEQ ID NOs:1-6 or functional variants thereof. The kit of parts may comprise three or more antigens according to SEQ ID Nos: 1-6 or functional variants thereof. The kit of parts may comprise peptide antigens according to each of SEQ ID NOs:1-6 or functional variants thereof.

For example, the kit of parts may comprise peptide antigens according to at least SEQ ID NO:3 or functional variants thereof.

In another example, the kit of parts may comprise peptide antigens according to at least SEQ ID NO: 3 or functional variants thereof and peptide antigens according to at least one of SEQ ID NOs:1-2 and 4-6 or functional variants thereof.

Other combinations of peptide antigens according to the second aspect of the invention are envisioned and included within the scope of the present invention.

The kit of parts may comprise one or more peptide antigens according to SEQ ID NOs:1-6 bound or anchored to a substrate. The substrate may be suitable for use in an immunoassay. The substrate may be a planar substrate such as a glass or plastic slide or similar, and the peptide antigens may be bound to one or both planar surfaces of the planar substrate. The substrate may be a reaction vessel or a wall of a reaction vessel. The substrate may be a well. The substrate may be a well plate and the peptide antigens may be bound to the surface of one or more wells of the well plate. The substrate may be a particle. Accordingly the peptide antigens may be bound to the surface of the particle. The particle may be a bead or similar. The particle may be an aggregate, or a crystalline material.

The one or more peptide antigens may be directly bound to the surface of the substrate. The N-terminus of the peptide antigen may be bound directly to the surface of the substrate. The C-terminus of the peptide antigen may be bound directly to the surface of the substrate.

The one or more peptide antigens may be bound indirectly to the substrate via a linker. The linker may space the one or peptide antigens from the surface of the substrate to increase the availability of the one or more peptide antigens for specific binding to the target antibody.

The substrate may be suitable for use in an immunoassay. Suitable immunoassays of the first aspect of the invention are suitable immunoassays of the present aspect of the invention. For example, the substrate may be suitable for use an enzyme-linked immunosorbent assay (ELISA). Accordingly the kit of parts may be used to determine whether a biological sample from a subject comprises the antibody for which the or each peptide antigen specifically binds. The presence of the antibody may be indicative that the subject from which the biological sample originates has a specific disease or medical condition. Therefore, the kit of parts may be used in an assay to determine whether a subject from which the biological sample is taken has that specific disease or medical condition.

The target antibody may be an antibody that specifically binds to a protein that is typically present in cellular membranes of at least one type of cancer cells. A biological sample comprising the target antibody may hinder or inhibit the activity of the protein to which it specifically binds. In embodiments where the target antibody binds to a protein that is present in the cellular membrane of at least one type of cancer cells, a biological sample comprising the target antibody may hinder or inhibit the growth of the of the at least one type of cancer cells.

The kit of parts may comprise a buffered solution within which the peptide antigens may be suspended. The kit of parts may comprise a washing buffered solution that may be used to wash a substrate to which the peptide antigens have been bound or anchored. The kit of parts may comprise a marker or label that may be used to mark or label antibodies that have bound to the peptide antigens during an immunoassay using the kit of parts.

Accordingly, the kit may allow the identification of biological samples that may be suitable for use as therapeutic agents for at least one type of cancer. A biological sample thereby identified may be processed to produce a medicament or pharmaceutical composition that may be given to treat the at least one type of cancer. The biological sample may be processed to produce a gamma globulin for cancer treatment, for example.

Processing of positively identified biological samples may comprise the extraction, concentration, or amplification of the target antibodies within the biological sample.

The invention extends in a fifth aspect to a composition comprising at least one antibody for fragment thereof that binds to the peptide antigen according to the second aspect of the invention.

Typically, the at least one antibody or fragment thereof is an IgG antibody.

The at least one antibody fragment thereof may be selected from the group: anti-VEGFR1a IgG, anti-VEGFR1b IgG, anti-FGFR2 IgG, anti-ERBB3 IgG, anti-ABCC3 IgG, or anti-ABCC5 IgG.

The at least one antibody fragment thereof may be selected from the group: anti-ERBB3 IgG, anti-ABCC3 IgG, anti-ABCC5 IgG, or anti-FGFR2 IgG.

The at least one antibody fragment thereof may be selected from the group: anti-ERBB3 IgG, anti-ABCC3 IgG or anti-FBGF2 IgG.

The composition may be a processed biological sample. The composition may be a blood plasma composition. The composition may be a purified blood plasma composition. For example, the composition may be blood plasma that has been screened to remove platelets, virus particulates or similar.

Alternatively, the composition may arise from an alternative aqueous solution comprising at least one antibody or fragment thereof. For example, the at least one antibody or fragment thereof may be purified and separated from a biological sample and re-suspended in suitable aqueous medium. The suitable aqueous medium may be a buffered aqueous solution.

Accordingly, the composition of the present aspect may be a synthetic composition that does not occur naturally.

According to a sixth aspect of the invention there is provided the use of the composition according the fifth aspect for the treatment of cancer.

Preferably, the composition comprises a therapeutically effective amount of the at least one antibody or fragment thereof that binds to the or each peptide antigen according to the second aspect.

Administration of the composition to a subject may allow the antibody to bind to the target cell membrane protein to thereby inhibit the activity of that protein, thereby slowing or preventing the growth of the host cell. The composition may be administered directly to the specific site to be treated. The composition may be administered to the subject generally. For example, where the composition is a blood plasma composition, the composition may be used in a transfusion or injection into a subject for the treatment of cancer.

Preferred and optional features of the first to sixth aspects are preferred and optional features of the first to sixth aspects. In other words, features disclosed for each aspect may be taken to be a feature for any of the other aspects.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### ELISA Kit for Cancer Treatment

The linear peptide antigens of the invention were synthesised by a solid-phase chemistry with 95% purity, and then used to develop an in-house ELISA for detection of anticancer IgG antibodies in human plasma. The plasma rich in anticancer IgG antibodies against individual peptide antigens was then used to grow cell lines derived from various cancers. We finally selected the linear peptide antigens that could bind to anticancer IgG to develop an ELISA antibody test kit with a mixture of the peptide antigens of the invention.

### Plate Coating

96-well plates, acting as substrates according to the invention, were prepared for use in an ELISA-type assay as follows:

a. Materials
96-well Maleimide Activated Plates (15150, Thermo Scientific).
1M phosphate buffer (p3619, Sigma-Aldrich).
Binding Buffer: 100 mL: 0.1M phosphate buffer containing 0.15M NaCl and 10 mM EDTA, pH 7.2: (prepared using 10 mL 1M phosphate buffer +0.85g NaCl+292mg EDTA+90 mL deionized water)
Wash Buffer: 200 mL: 0.1M phosphate buffer containing 0.15M NaCl and 0.05% Tween 20, pH 7.2: (prepared using 20 mL 1M phosphate buffer+1.7 g NaCl+0.1 mL Tween 20+180 mL deionized water).
Cysteine-HCl (C1276-10G, Sigma-Aldrich): 10 μg/mL.
Synthetic peptide antigens: 5 mg/mL 67% acetic acid.
   b. Coating Procedure
Wash microplate three times with 200 μL Wash Buffer before use.
Prepare antigens at 20 μg/mL in Binding Buffer.
Add 100 μL antigen working solution to each well and incubate at 4° C. overnight.
Wash the plate three times using 200 μL Wash Buffer.
Immediately before use, prepare cysteine solution at 10 μg/mL in Binding Buffer. Add 200 μL to each well and incubate for 1 hour at room temperature to deactivate excess maleimide groups.
Wash the plate twice using 200 μL of Wash Buffer before drying at 40° C.
Seal the dried plates with foil film and keep them at 2-8° C. up to 6 months.

### ELISA Protocol for Detection of Anticancer IgG Antibodies

A typical ELISA protocol that is suitable for use with the peptide antigen of the invention is provided below.

Human plasma samples were purchased from Biosciences (Cambridge, UK), which were collected from healthy blood donors. Pooled plasma of 20 randomly selected plasma samples was used as a reference sample (RS) to detect plasma either rich or absent in anticancer IgG antibodies on each 96-well plate. In brief, Maleimide-activated plates (Cat. 15150, Thermo Scientific, Edinburgh, UK) were coated based on the Manufacturer's instruction. The antigen-coated plate was washed twice with 200 μL Wash Buffer that was phosphate-buffered saline (PBS) (P4417, Sigma-Aldrich, Ayrshire, UK) containing 0.05% Tween-20; 50 μL plasma sample diluted 1:200 in Assay Buffer that was PBS containing 0.5% bovine serum albumin (BSA) was then added to each sample well; 50 μL Assay Buffer was added to each negative control (NC) well and 50 μL RS sample was added to each RS well. Following incubation at room temperature for 1.5 hours, the plate was washed three times with 200 μL Wash Buffer and 50 μL peroxidase-conjugated goat anti-human IgG antibody (ab98567, Abcam, Cambridge, UK) diluted 1:30000 in Assay Buffer was added to each well. After incubation at room temperature for 1 hour, colour development was initiated by adding 50 μL Stabilized Chromogen (SB02, Life Technologies, Warrington, UK) and terminated after 20 minutes by adding 25 μL Stop Solution (SS04, Life Technologies). The measurement of optical density (OD) was completed on a microplate reader within 10 minutes at 450 nm with a reference wavelength of 620 nm.

### Calculation of SBR

All the samples will be tested in duplicate and the specific binding ratio (SBR) can be used to represent the relative levels of plasma IgG antibodies. The SBR is calculated as follows:

$$SBR = \frac{( {OD}_{Sample} - {OD}_{NC} )}{{OD}_{Control} - {OD}_{NC}}$$

where ODₛₐₘₚₗₑ is defined as OD measured for plasma rich in anticancer IgG antibodies, OD_{NC} is defined as OD measured for the negative control, and OD_{control} is defined as OD measured for the pooled reference sample.

Samples with a high SBR are identified as samples comprising significant levels of anticancer IgG antibodies.

Accordingly, blood plasma samples comprising significant levels or concentrations of the target antibodies that bind to the peptide antigens of the invention can be identified using the above assay. As a result identified samples can be used to generate potential therapeutics for the treatment of cancer. The efficacy of identified blood plasma samples may be investigated by testing the effect of the blood plasma samples on cancer cell line proliferation. Table 3 gives the relative levels of anticancer IgG against the peptide antigens listed in Table 1.

TABLE 3
Relative levels of anticancer IgG antibodies in human plasma

| SEQ ID | Target | Positive | Negative | Ratio of |
|---|---|---|---|---|
| NO: | protein | levels (SBR) | levels (SBR) | positive/negative |
| 1 | VEGFR1a | 1.71 | 0.24 | 7.1 |
| 2 | VEGFR1b | 4.56 | 0.11 | 41.5 |
| 3 | FGFR2 | 1.07 | 0.06 | 17.8 |
| 4 | ERBB3 | 0.32 | 0.06 | 5.3 |
| 5 | ABCC3 | 1.07 | 0.16 | 6.7 |
| 6 | ABCC5 | 0.83 | 0.12 | 6.9 |

### Cell Proliferation Assay

Four cell lines derived from hepatocellular carcinoma (HCC) and pancreatic adenocarcinoma (PA) were purchased from the European Collection of Authenticated Cell Cultures (ECACC), Porton Down, UK. Of these four cancer cell lines, two were derived from human HCC including Hep B3 and Huh-7D12, AsPC-1 from human pancreas adenocarcinoma ascites metastasis and BxPC-3 from human primary pancreatic adenocarcinoma. These cancer cell lines were seeded in a 96-well plate, 100 μL/well with a density of 2.5×10⁴ cells/ml RPMI 1640 medium (Gibco, USA) containing 10% fetal calf serum (FCS), and cultured in humidified atmosphere with 5% CO₂ at 37° C. for 24 hours; these cancer cells with RPMI 1640 containing 20% human plasma either positive or negative for each anticancer IgG antibody for 48 hours, respectively, in the same conditions as mentioned above. Cell viability was assessed by the cell counting kit-8 (CCK-8, Sigma-Aldrich). Briefly, 10 μL CCK-8 solution was added to each well; after incubation at 37° C. for 2 hours, OD of each well was measured on a microplate reader at a wavelength of 450 nm. The complete medium was used as blank. Cell viability was used to present data and calculated as follows:

$$Cell viability = \frac{( {OD}_{positive} - {OD}_{blank} )}{( {OD}_{negative} - {OD}_{blank} )}$$

where ODₚₒₛᵢₜᵢᵥₑ is defined as OD measured in cancer cells cultured with plasma positive for anticancer IgG, and OD_{negative} is defined as OD measured in cancer cells cultured with plasma negative for anticancer IgG. Inhibitory rate was calculated with 1—cell viability. Cell viability data were expressed as mean ±standard deviation (SD), and either Student's t-test or Mann-Whitney U test was used to examine the difference in cell viability between cells treated with plasma positive for anticancer IgG and those treated with plasma negative for anticancer IgG. The P-value of <0.05 was considered to be statistically significant.

### Preliminary Results

Inhibitory effects of plasma anticancer IgG vary between different cancer cells. Cell viability of <0.9 with a P-value of <0.05 was defined as effective inhibition that may have therapeutic value for treatment of cancer. An inhibitory rate was also used to present data, which was defined as (1—cell viability)×100%.

As shown in Table 4, anti-ERBB3 IgG had an inhibitory rate of 29% for proliferation of Hep B3 cells (P=0.003) and 17% for Huh-7D12 cells (P<0.001).

TABLE 4
Inhibitory effect of plasma anti-ERBB3 IgG on proliferation
of cancer cells

| Anti- | Hep B3 | Huh-7D12 | AsPC1 | BxPC3 |
|---|---|---|---|---|
| ERBB3 | mean ± | mean ± | mean ± | mean ± |
| IgG | SD (n) | SD (n) | SD (n) | SD (n) |
| Positive | 0.71 ± 0.13 (9) | 0.83 ± 0.09 (9) | 1.22 ± 0.27 (9) | 1.05 ± 0.12 |
| | | | | (9) |
| Negative | 1.00 ± 0.21 (9) | 1.00 ± 0.09 (9) | 1.00 ± 0.39 (9) | 1.00 ± 0.14 |
| | | | | (9) |
| Statistical | P = 0.003 | P < 0.001 | P = 0.186 | P = 0.440 |
| test | | | | |

As shown in Table 5, anti-ABCC3 IgG had an inhibitory rate of 24% for proliferation of Hep B3 cells (P=0.005) and 13% for Huh-7D12 cells (P=0.001).

TABLE 5
Inhibitory effect of anti-ABCC3 IgG on proliferation of cancer cells

| Anti- | | | | BxPC3 |
|---|---|---|---|---|
| ABCC3 | Hep B3 | Huh-7D12 | AsPC1 | mean ± |
| IgG | mean ± SD (n) | mean ± SD (n) | mean ± SD (n) | SD (n) |
| Positive | 0.76 ± 0.11 (9) | 0.87 ± 0.04 (9) | 0.92 ± 0.17 (9) | 0.96 ± 0.19 |
| | | | | (9) |
| Negative | 1.00 ± 0.20 (9) | 1.00 ± 0.09 (9) | 1.00 ± 0.22 (9) | 1.00 ± 0.14 |
| | | | | (9) |
| Statistical | P = 0.005 | P = 0.001 | P = 0.372 | P = 0.652 |
| test | | | | |

As shown in Table 6, anti-ABCC5 IgG had an inhibitory rate of 20% for proliferation of AsPC1 cells (P=0.047) and 24% for proliferation of Hep B3 cells (P=0.011 from Mann-Whitney U test).

TABLE 6
Inhibitory effect of anti-ABCC5 IgG on proliferation of cancer cells

| Anti- | | | | BxPC3 |
|---|---|---|---|---|
| ABCC5 | Hep B3 | Huh-7D12 | AsPC1 | mean ± |
| IgG | mean ± SD (n) | mean ± SD (n) | mean ± SD (n) | SD (n) |
| Positive | 0.76 ± 0.11 | 1.06 ± 0.19 | 0.80 ± 0.21 | 0.90 ± 0.12 |
| | (9) | (9) | (9) | (9) |
| Negative | 1.00 ± 0.39 | 1.00 ± 0.21 | 1.00 ± 0.20 | 1.00 ± 0.13 |
| | (9) | (9) | (9) | (9) |
| Statistical | P = 0.011 | P = 0.518 | P = 0.047 | P = 0.099 |
| test | | | | |

As shown in Table 7, anti-FGFR2 IgG had an inhibitory rate of 32% for proliferation of Huh-7D12 cells (P<0.001) and 23% for AsPC1 cells (P=0.004).

TABLE 7
Inhibitory effect of anti-FGFR2 IgG on proliferation of cancer cells

| Anti- | | | | BxPC3 |
|---|---|---|---|---|
| FGFR2 | Hep B3 | Huh-7D12 | AsPC1 | mean ± |
| IgG | mean ± SD (n) | mean ± SD (n) | mean ± SD (n) | SD (n) |
| Positive | 0.85 ± 0.09 (9) | 0.68 ± 0.07 (9) | 0.77 ± 0.18 (9) | 0.87 ± 0.11 |
| | | | | (9) |
| Negative | 1.00 ± 0.20 (9) | 1.00 ± 0.14 (9) | 1.00 ± 0.10 (9) | 1.00 ± 0.16 |
| | | | | (9) |
| Statistical | P = 0.06 | P < 0.001 | P = 0.004 | P = 0.057 |
| test | | | | |

As shown in Table 8, anti-VEGFR1a IgG had an inhibitory rate of 29% for proliferation of Hep B3 cells (P<0.001) and 19% for Huh-7D12 cells (P=0.016).

TABLE 8
Inhibitory effect of anti-VEGFR1a IgG on proliferation of cancer cells

| Anti- | | | | BxPC3 |
|---|---|---|---|---|
| VEGFR1a | Hep B3 | Huh-7D12 | AsPC1 | mean ± |
| IgG | mean ± SD (n) | mean ± SD (n) | mean ± SD (n) | SD (n) |
| Positive | 0.71 ± 0.12 (9) | 0.81 ± 0.14 (9) | 1.09 ± 0.29 (9) | 1.04 ± 0.12 |
| | | | | (9) |
| Negative | 1.00 ± 0.17 (9) | 1.00 ± 0.15 (9) | 1.00 ± 0.11 (9) | 1.00 ± 0.11 |
| | | | | (9) |
| Statistical | P < 0.001 | P = 0.016 | P = 0.537 | P = 0.421 |
| test | | | | |

As shown in Table 9, anti-VEGFR1b IgG had an inhibitory rate of 25% for proliferation of Hep B3 cells (P<0.001) and 21% for BxPC3 cells (P=0.009).

TABLE 9
Inhibitory effect of anti-VEGFR1b IgG on proliferation of cancer cells

| Anti- | | | | BxPC3 |
|---|---|---|---|---|
| VEGFR1b | Hep B3 | Huh-7D12 | AsPC1 | mean ± |
| IgG | mean ± SD (n) | mean ± SD (n) | mean ± SD (n) | SD (n) |
| Positive | 0.75 ± 0.08 (9) | 1.00 ± 0.17 (9) | 1.10 ± 0.21 (9) | 0.79 ± 0.11 |
| | | | | (9) |
| Negative | 1.00 ± 0.10 (9) | 1.00 ± 0.19 (9) | 1.00 ± 0.17 (9) | 1.00 ± 0.18 |
| | | | | (9) |
| Statistical | P < 0.001 | P = 0.968 | P = 0.274 | P = 0.009 |
| test | | | | |

In summary, all of the six antibodies detected using the peptide antigens of the invention demonstrated inhibition of cancer cell proliferation in the above experiments for at least one cancer cell type. Anti-FGFR2 IgG showed the most powerful inhibiting effects on the growth of cancer cells of the 6 distinct antibodies tested; the IgG antibodies against the other five antigens can also inhibit the growth of cancer cells in various degrees.

1. A method of determining whether a biological sample comprises target antibodies that have anticancer activity, the method comprising the steps:
i) providing a biological sample;
ii) providing at least one peptide antigen according to any one of SEQ ID NO:1-SEQ ID NO:6 or functional variants thereof;
iii) contacting the biological sample to the at least one peptide antigens;
iv) determining the concentration of target antibodies that were present in the biological sample that bind specifically to the peptide antigens; and
v) comparing the determined concentration of target antibodies present in the biological sample to a reference concentration,
wherein a significant increase in the concentration of target antibodies in the biological sample compared to the reference concentration is indicative that the biological sample comprises a significant concentration of the one or more target antibodies that have anticancer activity.

2. The method of claim 1, wherein the or each peptide antigens are anchored to a substrate.

3. The method of claim 1, wherein the biological sample is a sample of whole blood or a sample of a blood fraction, such as blood plasma, or blood serum.

4. The method of claim 1, wherein the method comprises providing peptide antigens according to at least SEQ ID NO: 3 or functional variants thereof.

5. The method of claim 1, wherein the method comprises the step of applying a label to target antibodies that have specifically bound to the peptide antigens.

6. The method of claim 5, wherein the label is conjugated to a secondary antibody.

7. The method of claim 6, wherein the method comprises the step of adding an antibody enzyme conjugate that is configured to bind to any antibodies that have bound to the peptide antigen.

8. The method according to claim 5, wherein the method comprises the step of washing after the application of a label to remove label that is not applied or bound to a target antibody.

9. A peptide antigen corresponding to SEQ ID NOs:1 to 6 or functional variants thereof.

10. The use of peptide antigens of claim 9 to produce antibodies or aptamers for the treatment of cancer.

11. A kit of parts comprising one or more peptide antigens according to SEQ ID NOs: 1 to 6 or functional variants thereof.

12. The kit of parts according to claim 11 comprising peptide antigens according to at least SEQ ID NO:3 or functional variants thereof.

13. The kit of parts of to claim 11, wherein the one or more peptide antigens are bound to the surface of a substrate.

14. The kit of parts according to claim 13, wherein the one or more peptide antigens are bound directly to the surface of the substrate.

15. The kit of parts according to claim 13, wherein the one or more peptide antigens are bound indirectly to the surface of the substrate via a linker.

16. A composition comprising at least one antibody that binds to the peptide antigen according to any one of SEQ ID NOs:1-6 or functional variants thereof.

17. The composition of claim 16 comprising at least one antibody that binds to the peptide antigen according to SEQ ID NO:3 or functional variants thereof.

18. A method of treating cancer, the method comprising administering to a subject a therapeutically effective amount of the composition according to claim 16.

19. A method of purifying anticancer gamma-globulin, the method comprising providing human plasma comprising a therapeutically effective amount of the composition according to claim 16, thereby purifying anticancer gamma-globulin.

20. A method of treating cancer, the method comprising administering to a subject a therapeutically effective amount of anticancer gamma-globulin purified according to claim 19.
